(19) United States
(12) Patent Application Publication
(10) Pub. No.: US 2016/0262626 A1
(43) Pub. Date: Sep. 15, 2016

(54) DEVICE FOR NON-INVASIVE DETECTION OF PREDETERMINED BIOLOGICAL STRUCTURES

(71) Applicant: INSONO S.R.L., Sesto Fiorentino (FI) (IT)

(72) Inventors: Giulio PELOSI, Firenze (IT); Gionatan TORRICELLI, Firenze (IT); Fabio CIORIA, Firenze (IT); Leonardo MASOTTI, Sesto Fiorentino (IT)

(21) Appl. No.: 15/031,461

(22) PCT Filed: Oct. 21, 2014
(86) PCT No.: PCT/IB2014/065506

§ 371 Date: Apr. 22, 2016

(30) Foreign Application Priority Data
Oct. 23, 2013 (IT) FI2013A000255

(51) Int. Cl. A61B 5/00

(52) U.S. Cl. CPC A61B 5/0082; A61B 5/0062; A61B 5/742; A61B 5/0077; A61B 5/489; A61B 2576/00

(57) ABSTRACT

Device for non-invasive detection of predetermined biological structures associated with areas visible to the naked eye, provided with a) means for illuminating the area to be investigated by means of infrared light, adapted to be absorbed selectively by the biological structure to be investigated, and light with band in the visible field, b) means for splitting the light at the inlet to the cameras, into a light beam with light with spectral band relating to the infrared field and light beam with spectral band in the visible c) acquisition means of two images, one relating to the information content of the split light beam relating to the infrared field, and one relating to the information contribution of the light beam in the visible field, d) means for superimposing the two images to produce a single image that shows the area to be investigated as in the visible with superimposed the shape of the hidden structure to be investigated, e) a viewer for viewing said single image.

### TECHNICAL FIELD

The present invention relates to the field of diagnostic or operational investigations of biological structures present in the human body, and more in particular relates to a device for non-invasive detection of predetermined biological structures of interest, such as preferably, but not exclusively, hidden biological structures, such as surface blood vessels. The present invention also relates to a method for non-invasive detection of predetermined biological structures, for example implemented by means of this device.

### STATE OF THE ART

As is known, an image (fixed, such as a photograph, or a video, i.e. a sequence of “frames”) is the result of the action of the light (i.e. an electromagnetic radiation) coming from a source on the objects of the scene being viewed and of the method with which the light, from these objects, “returns” toward the image acquisition sensor, which can be the human eye, an optical sensor, etc. According to the physical characteristics of the electromagnetic wave emitted by the source, i.e. of its wavelength (or more probably, a spectral band of wavelengths, as in practice it is always a beam of electromagnetic waves that is emitted and that, even for radiations with a very narrow band, have a slight variability in their wavelengths), and to the way with which the real scene (the real structures that “occupy” or “form” the scene) that is struck by the electromagnetic wave (more tangibly, a band of electromagnetic waves) “interacts” with the wave and the consequent method (wavelength, intensity, inclination etc.) with which the sensor receives the electromagnetic waves coming from the real scene (generally varied with respect to those sent by the source, as they interact with this scene), the image supplied by the sensor will be provided with different information contributions, i.e. will give different information to the person viewing the image. For example, if a given scene is illuminated only by a light with spectral band in the visible and the scene is viewed by a sensor sensitive to visible light and to infrared light, the image produced by the sensor will relate to what is visible and the relevant information content associated with the image will relate to what is viewed in the image and correlated with the visible band. Instead, if the same scene is illuminated only with a light with spectral band only in the infrared, the image produced by the same sensor will be different with respect to that in the visible and the relevant information content associated with the image will differ from the previous information content.

The term information contribution is intended as everything associated with what is seen in the image in relation to the spectral bands of the returning light beams that struck the sensor; for example, in the case of the visible, contributions relating to space, shape, color, etc. can be present while in the case of the infrared, information relating to space and shape can be present, but for example relating to “structures” not visible to the naked eye (owing to the greater penetrating power of infrared radiation) and also information relating to the temperature of the structures based on the intensity of the radiation emitted. In general, each body and substance has an absorption spectrum, i.e. a window of frequencies in which a white light is reflected with less intensity due to the interaction of those wavelengths with the specific substance. This is the physical principle underlying all spectroscopic instruments that are capable of obtaining information on the substances that form the sample highlighting the bands in which the light scattered by the sample is greatly attenuated following illumination due to absorption of the light used as illumination by the componing substances.

Different substances have absorption spectra in the ultraviolet, in the visible or in any part of the infrared. For this reason, having several images corresponding to different optical bands makes it possible to obtain information regarding different structures forming the scene whose composition differs from the others.

Following this principle, the devices for non-invasive detection of the surface blood vessels in order to facilitate taking blood samples or in any case interaction with the venous tissues, are based essentially on three operating principles: reflection of light with wavelengths or spectral bands in the NIR (Near InfraRed) field by tissues not supplied with blood, transillumination with visible light, and transillumination with NIR light.

The transillumination devices with NIR light project an NIR light beam transversely with respect to a receiver. These devices operate in contact with the tissue to be investigated showing the underlying vein on a display positioned on the device. A red laser beam is emitted from the side of the device to indicate to the physician where to inject. This point is not viewed on the display but is extrapolated based on the direction of the vein in the point in which it exits from the screen. From an operational viewpoint, although it seems that viewing is particularly detailed, this device is awkward to use as, being in contact with the patient, it must be continuously sterilized for subsequent uses or an interchangeable protection must be applied. Moreover, it is particularly awkward for pediatric use, due to the limited size of the limbs and, consequently, of the veins.

NIR reflection devices instead detect buried structures in general (blood vessels) using reflected NIR light and exploiting a method of projecting the hidden structures onto the surface of the object being analyzed (patient) through visible light. In practice, the hidden structures are scanned using NIR light and their form is subsequently projected by the device onto the surface that conceals them, for example the patient's skin.

From a patent viewpoint, there are various patents dealing with the problem of detecting biological structures hidden by the skin surface of a patient.

For example, in U.S. Pat. No. 4,817,622, a part of the body, typically the inside of the elbow, is illuminated with an infrared light source. A camcorder for producing video images, with which a monitor positioned immediately above the camcorder is associated, is used to observe the skin. The camcorder is sensitive to infrared radiation. The image shows only the parts that absorb the infrared light, such as the veins with respect to the parts of flesh, which are illuminated to a greater extent. There is also a circuit for amplifying the signal and increasing the contrast of the infrared image. This solution is limited, as it only allows the venous structure to be viewed and is unable to help the operator to understand where the venous structure is located with respect to the outer surface.

U.S. Pat. No. 6,032,070 describes a method and a system for viewing an anatomical structure such as blood vessels in high contrast with respect to the surrounding tissue. The system provides for the integration of a system for illuminating and receiving the infrared image in a helmet. The system is provided with systems and methods for supplying an anatomical image of the structures with enhanced contrast.

### OBJECT AND SUMMARY OF THE INVENTION

The object of the present invention is to produce a device for non-invasive detection of predetermined biological structures of interest, such as preferably, but not exclusively, surface blood vessels, which facilitates its use for the operator, giving him/her a feeling of “augmented reality” in viewing the biological structures of interest.

Another important object of the present invention is that of producing a device for non-invasive detection of predetermined biological structures of interest, reducing to a minimum contact between device and patient during use.

Yet another important object of the present invention is that of producing a device for non-invasive detection of predetermined biological structures of interest, which can be used easily of any part of a patient.

These and other objects, which will be more apparent below, are achieved with a device for non-invasive detection of predetermined biological structures of interest, such as preferably, but not exclusively, hidden biological structures like surface blood vessels as indicated in the appended claim 1.

According to a first aspect, the invention relates to a device for non-invasive detection of predetermined biological structures of interest, preferably of hidden type, associated with areas visible to the naked eye, comprising:
at least one light source adapted to illuminate the area to be investigated by means of one or more first light beams with spectral bands included in a spectral band subject, in a predetermined manner, to absorption and/or reflection and/or to other interaction typical of the particular hidden biological structure to be detected in order to detect at least specific parameters of said biological structure, among which at least the shape and position in space,
an image acquisition unit with field of view directed at said area to be investigated, adapted to acquire images associated with one or more light radiations coming from said area to be investigated, following illumination by said at least one light source and in particular images with which there are associated at least two groups of information contributions, namely
   a first group of information contributions relating at least to the hidden biological structure, i.e. information contributions associated with at least one first light radiation with spectral band corresponding to a field such that a light radiation is capable of being scattered, reflected or absorbed by the biological structure of interest in a manner differing at least in part from the surrounding biological tissues and/or structures, so that said images are capable of showing said biological structure of interest,
   a second group of information contributions differing at least in part from said first group of information contributions, relating to another or other biological structures interacting differently with said first light radiation with respect to said biological structure of interest to be investigated, present in the field of view of said image acquisition unit,
electronic means adapted to
   process at least said first group of information contributions relating to the biological structure of interest, and to
   combine in at least one image the processed information contributions of said first group relating to the biological structure of interest and the information contributions, optionally also processed, of said second group relating to said another or other biological structures in the field of view of said image acquisition unit,
at least one viewer, adapted to allow viewing of at-least said image, so that the user of the device, with said device at a distance from said area to be investigated and observing said at least one viewer, sees continuously at least one processed image of said biological structure of interest combined with an image of said another or other biological structures present in the same field of view.

The term “typical interactions” of the tissue relates to all the possible known primary optical interactions, among which absorption, reflection, scattering that can differentiate the behavior of the specific tissue subjected to investigation from the surrounding tissues or which can provided information constituting or relating to the state of development and/or aging and/or other biological, anatomical and pathological factors. Among the typical interactions it is also possible to include other specific interactions resulting from the absorption of a given electromagnetic radiation, among which phosphorescence and fluorescence and similar, and/or combinations of these. Also in these cases, it is possible to obtain information linked to the parameters mentioned above, linked to the state of the tissue subjected to investigation in a specific manner with respect to other surrounding tissues that can, even only partly, hide and/or surround it.

The term biological structure is intended, for example, as any cellular conformation of the human body. For example, the epidermis is considered as a biological structure, just as a group of tumor cells, although not having a defined “structure” is also considered, in the present invention, as a biological structure. Consequently, this term can be intended as a clearly defined structure of the human body or also, more generally, as a portion characterized by biological activity.

Hereinafter, “hidden” biological structure or “hidden” structure is intended as any biological structure, such as a tissue or any combination of tissues, not completely or partly visible during conventional visual inspection. In particular, it may not be completely visible as it underlies other tissues or because its dimensions are not compatible with detection by the naked eye. For example, hidden structure can refer to the assembly of tissues that produce a venous vessel composed at least of the endothelium, of the sub endothelial connective tissue, of the smooth vascular muscle and of the blood. This structure is often almost completely hidden by the tissues of the epidermis, except for the surface vessels. The deeper this type of structure is with respect to the overlying tissue, the more it is hidden, as the visible light is unable to penetrate these external tissues. Other biological structures are partly hidden, as not all their parts are completely subject to visual analysis. For example, for some skin diseases such as ulcers, conventional visual analysis can only be carried out on the outer layers but it is not possible to obtain any information regarding the innermost layers and their nature (arterial or venous) cannot be detected. Moreover, even in the outermost parts it is not possible to detect a series of physiological and pathological parameters through visual analysis, as they are not correlated to the response of visible light, but only to other types of radiation. Moreover, other structures such as malignant neoformation of the skin (melanoma), cannot be completely detected through visual inspection, both because the innermost layers are not accessible by visible light, but also because some of its microscopic substructures (that characterize it at anatomo-pathological level) cannot be detected by the naked eye. Moreover, in this type of conventional analysis the degree of vascularization, the degree of infiltration and other factors that are of great help for the diagnosis and planning of any curative and surgical interventions cannot be detected.

The present invention relates to biological structures, defined “of interest”, which may also not necessarily be “completely hidden”, i.e. structures for which simple inspection through illumination with visible light does not allow detection of all the desired information.

According to embodiments of the invention, the information contributions of said second group of information contributions can relate to the surface contours and/or to surface elements of the area to be investigated, such as to define the external shape and the position in space of the area to be investigated, so that the images containing said information contributions of the second group are capable of showing the surface contour and/or other surface elements of said area to be investigated.

According to embodiments of the invention, preferably the electronic means of the device are adapted to process at least said first group of information contributions relating to the biological structure of interest, in this specific case they are programmed to increase/highlight the part relating to the shape of said biological structure of interest; preferably acquisition, processing and combination of said images containing said information contributions of said first and second group take place in real time.

According to preferred embodiments, the device according to the invention comprises splitting means adapted to split the scattered and/or reflected light beams coming from said area to be investigated, following illumination by said at least one light source, into at least two distinct light radiations, respectively at least one first light radiation with a first spectral band corresponding to a field such that a light radiation is capable of being scattered, reflected or absorbed by the biological structure of interest in a manner differing at least in part from one or more surrounding biological structures, and at least one second light radiation with second spectral band differing at least in part from said first spectral band, capable of being scattered, reflected or absorbed by said biological structure of interest in a manner differing at least in part with respect to said first light radiation; the image acquisition unit is adapted to acquire images respectively associated with said light radiations split by the splitting means, i.e. respectively with said at least one first light radiation and with said at least one second light radiation, and in particular at least one first image with information contributions belonging to said first group of information contributions relating to the biological structure of interest and at least one second image with information contributions relating to said second group of information contributions; said electronic means being adapted to combine said
at least one first image acquired, or processing of said at least one first image acquired, containing information contributions relating to the biological structure of interest and
at least one second image acquired, or processing of said at least one first [second] image acquired, containing information contributions of said second group of information contributions.

In practice, according to this alternative embodiment, the light beams coming from the area to be investigated can be split into two or more series of light radiations with different spectral bands, and these series can be detected in a manner separate from one another by the image acquisition unit, thus obtaining images associated with the type of spectral band of the specific series. These images can be processed, or not, and combined with one another to obtain the desired result.

Preferably, said at least one second light radiation has a spectral band included at least in part in the spectral band of the visible, so that said image acquisition unit is adapted to acquire images respectively associated with said light radiations split by said splitting means, i.e. respectively with said at least one first light radiation and with said at least one second light radiation with band in the visible, and in particular at least one first image with information contributions belonging to said first group of information contributions relating to the biological structure of interest and at least one second image with information contributions relating to the surface view of the area to be investigated; the viewer is therefore capable of viewing, preferably in a single image, the area to be investigated from the outside, i.e. as visible to the naked eye, and simultaneously the biological structure (or structures) of interest present in the same area, which would not be visible to the naked eye. With this solution an optimal “augmented reality” is obtained, which facilitates the work of a healthcare operator. For example, in the case of an operator requiring to find the vein in a patient's arm, the device is capable of displaying, on the viewer, both the arm in its “real” view, i.e. as if it were visible to the naked eye, and the veins, in their real position inside the arm.

According to preferred embodiments, the electronic means, which are adapted to process at least said first group of information contributions relating to the biological structure of interest, in particular are programmed to increase/highlight the part relating to the shape of said biological structure of interest; preferably acquisition, processing and combination of said images containing said information contributions of said first and second image group takes place in real time.

Preferably, the device comprises at least two light sources, respectively
at least one said first light source adapted to illuminate the area to be investigated, visible to the naked eye, by means of one or more of said first light beams,
at least one second light source for one or more second light beams with spectral bands differing at least in part from the bands of said first light beams emitted by said first source; the second light beams are adapted to interact optically with the area to be investigated in a different manner with respect to said first light beams and to produce images containing said second group of information contributions.

Preferably, said light beams of said first and said second source are emitted substantially simultaneously from said two sources.

In this way, the “dedicated” illumination of the area to be investigated with different lights with different spectral bands makes it possible to analyze different biological structures that interact in a differentiated manner to the specific types of light that strike them, obtaining specific images with information contributions differentiated from one another according to the type of light, which can then be combined with one another to give the desired output.

According to preferred embodiments, the second light source emits one or more second light beams with spectral bands at least in the visible. In this way, the “dedicated” illumination of the area to be investigated with light with spectral band in the visible allows optimal yield of the image acquisition with information contributions in the visible, regardless of the ambient lighting conditions outside the device.

In preferred embodiments, the device comprises means for adjusting the luminosity and/or power of said at least one light source. This adjustment can take place “automatically”, in the sense that the device can be capable of evaluating, based on the ambient lighting conditions, and/or on the distance from the area to be investigated and/or based on the anatomy and/or surface color of the area to be investigated, the necessary luminosity and/or power of said at least one light source (and in particular of the source of visible light, if present).

According to preferred embodiments, the at least one said light source comprises a planar distribution of LEDs.

According to preferred embodiments, each light source is made more homogeneous in the viewing area through appropriate scattering means interposed between it and the space subjected to investigation.

Preferably, the image acquisition unit acquires said at least one first and at least one second image in a substantially simultaneous manner.

According to preferred embodiments, the image acquisition unit comprises at least two distinct image acquisition cameras, at least one first camera to acquire at least one first image deriving from said at least one first light radiation split by said splitting means and associated with the viewing of said biological structure of interest, and at least one second camera to acquire at least one second image deriving from said at least one second split light radiation; preferably said at least one second camera is adapted to acquire at least one second image deriving from said at least one second light radiation having spectral band preferably at least in part in the visible, associated with the viewing of the surface structures of the area to be investigated visible to the naked eye; said cameras preferably being image sensors.

Preferably, the device comprises at least one optical unit through which to receive the light radiation coming from said area to be investigated, said optical unit comprises said splitting means and said at least two cameras arranged so as to observe the same field of view. Preferably, this optical unit defines optical paths, i.e. routes which from the area to be investigated reach corresponding image acquisition cameras, which all have the same optical length.

Preferably, said optical unit comprises at least one optical focusing system arranged at the inlet to said optical unit or at the inlet to one or both said cameras; preferably said optical focusing system comprises at least one zoom system.

According to embodiments, the device comprises at least one single optical unit through which it receives all the light radiation coming from said area to be investigated; this single optical unit comprises said splitting means and said at least two cameras arranged so as to observe the same field of view.

According to preferred embodiments, the device comprises a filter arranged between said splitting means and said first camera adapted to permit the passage, toward said first camera, of at least one light beam with wavelength or spectral band corresponding to a field such that a light radiation is capable of being scattered, reflected or absorbed by the hidden biological structure of interest in a manner differing from the surrounding biological structures, preferably approximately in the infrared field between around 700 nm and 1000000 nm, and more preferably between 700 nm and 1000 nm.

According to preferred embodiments, the device comprises a filter arranged between said splitting means and said second camera adapted to allow at least one light beam with wavelength or spectral band belonging to the visible field, to pass toward said second camera.

Preferably, the splitting means, adapted to split the light radiation coming from said area, comprise at least one mirror adapted to reflect light radiation belonging to a predetermined spectral band toward predetermined image acquisition areas and to transmit the other light radiations belonging to all the other spectral bands toward other image acquisition areas.

Preferably, the splitting means, adapted to split the light radiation coming from said area, comprise a beamsplitter or the like, interposed between optical inlet of the device and image receiver unit, which allows transmission of the light beam with wavelength in the visible toward said second camera and reflection of the beam with wavelength in the infrared toward said first camera; preferably said beamsplitter comprising a cube beamsplitter or a prism beamsplitter or a three-band beamsplitter, or a hot-mirror, or the like.

Preferably, the device comprises a polarizer at the inlet to the optical unit.

In preferred embodiments, the device comprises two opposed main faces, a first face in which said viewer is provided and a second opposite face in which the optical inlet of said image acquisition unit is arranged. Advantageously, the majority of the electronic managing, processing and viewing components of the device are substantially comprised between said two faces, the extension in space of the device being essentially flat.

Preferably, the viewer is a touch-screen monitor; preferably, the controls for managing the device are prevalently managed from the touch-screen interface of the monitor.

According to preferred embodiments, the device comprises a supporting arm connected to a support structure for supporting the main part of the device comprising said viewer and said image acquisition unit, so that said main part can be positioned stably in space, above the detection area; preferably there being integrated in said supporting arm an electrical connection of said main part to an electrical power source; preferably said support structure is a base or a device for reversible fixing to a load-bearing structure. Preferably, this supporting arm is of jointed type; preferably, the arm has at least one joint of motorized type; preferably, the motorized movement of said at least one joint is managed from said touch-screen.

Advantageously, with this arm, there can be present reversible connection means between said arm and said main part of the device comprising said viewer and said image acquisition unit; there is associated with said main part a battery for autonomous operation when separated from the arm; preferably said reversible connection means comprise an apparatus for locking said main part to said arm, preferably with motorized operation.

Advantageously, this support structure can be a cart that moves on the ground; preferably, said cart comprises a surface for resting the part of the patient's body with the area to be investigated; preferably said resting surface has a concavity facing upward and elongated longitudinally to receive a portion of the patient's arm.

According to preferred embodiments, the at least one first light beam emitted by said at least one light source, adapted to be absorbed in a predetermined manner by the biological structure of interest to be detected, has a first wavelength and/or a first spectral band selected in a field of wavelengths between around 700 nm and 1000000 nm, and more preferably between around 700 nm and 1000 nm, i.e. preferably approximately in the infrared field; preferably the at least one light beam emitted by said at least one second light source or emitted by the environment has a wavelength or spectral band between around 300 nm and 800 nm, i.e. approximately in the visible field.

Preferably, the at least two image acquisition cameras comprise respective image sensors having adequate sensitivity in the bands of the optical radiations they are responsible for acquiring; preferably, these sensors operate in a spectral band between 10 nm and 1 mm and more preferably between 300 nm and 1000 nm.

According to another aspect, the invention relates to a method for non-invasive detection of blood vessels in proximity of the body surface, obtained with a device according to one or more of the preceding embodiments or with another device. This method provides for:
illuminating the area to be investigated by means of one or more light beams with wavelengths or spectral bands included in a spectral band subject to absorption in a manner predetermined by the hidden biological structure to be detected, and with one or more light beams with wavelength or spectral band in the visible,
splitting the light beams scattered and/or reflected coming from said area to be investigated, following said illumination, into at least two distinct light radiations, respectively at least one first light radiation with wavelength or spectral band corresponding to a field such that a light radiation is capable of being scattered, reflected or absorbed by the hidden biological structure in a manner differing from the surrounding biological structures, and at least one second light radiation with wavelength or spectral band included at least in part in the spectral band of the visible,
acquiring images respectively associated with said split light radiations, i.e. respectively with said at least one first light radiation and with said at least one second light radiation, and in particular at least one first image with information contributions relating to the hidden biological structure and at least one second image with information contributions relating to the surface view of the area to be investigated,
superimposing said
   at least one first image acquired, or processing of said at least one first image acquired, containing information contributions relating to the hidden biological structure and
   at least one second image acquired, or processing of said at least one second image acquired, containing information contributions relating to the surface view of the area to be investigated, to produce a single output image that includes both the image of the information contributions relating to the hidden biological structure, and the image of the information contributions relating to the surface view of the area to be investigated,
viewing said single image on at least one viewer, so that a user observing said at least one viewer sees the area to be investigated and, superimposed on this area, the shape of said biological structure.

Preferably, the method also comprises an electronic processing step of said at least one first image and/or of said at least one second image, adapted to modify said at least one image before the superimposing step; preferably said processing step being adapted to increase/highlight the part relating to the shape of said hidden biological structure; preferably acquisition, processing and superimposing of said images taking place in real time.

Preferably, the method also comprises a step of adjusting the luminosity and/or power emitted toward said area to be investigated.

Preferably, the method also comprises a step of focusing, manual or automatic, of said area to be investigated by an optical acquisition unit of said images.

Preferably, the method comprises one or more devices for polarizing the light at the inlet to said optical unit.

Preferably, the method comprises a step of filtering respectively in the field of the infrared and of the visible for said split light beams.

According to another aspect, the invention relates to a method for non-invasive diagnosis of various kinds of skin diseases or changes, such as bed sores, venous and/or arterial ulcers to a wide variation of skin diseases (mycosis, dermatitis, moles, melanoma, etc.) and of the surface tissues, whether they are accessible from the outside of the body and/or completely or partially by intracavitary means. This method provides for:
illuminating the area to be investigated by means of one or more light beams belonging to spectral bands that allow at least one typical interaction of the tissue investigated with this light,
splitting the light beams received from the same optic as response of the tissue based on the optical interaction that took place in said area to be investigated, following said illumination, into all the bands that allow diagnostic parameters of the tissue investigated to be correlated with the images deriving from acquisition of the response of the tissue in the specific band,
acquiring images respectively associated with said split light radiations,
processing said multiple images in combined manner so as to extrapolate global and/or local information regarding the biological, anatomical and pathological parameters starting from the hyperspectral information consisting of the multiple images deriving from acquisition of the light bands described above,
viewing this diagnostic information in an appropriate form for the specific case, for example through a single image on at least one viewer, so that a user viewing said at least one viewer sees the area to be investigated and, superimposed on this area, the shape of said biological structure and specific colorings correlated to the parameters detected, or through multiple simultaneously presented images that provide different information, or through other real time viewing techniques that are capable of supplying the user with information relating to the aforesaid parameters that are not visible through simple analysis to the naked eye.

### BRIEF DESCRIPTION OF THE DRAWINGS

Further characteristics and advantages of the invention will become more apparent from the description of several preferred but non-exclusive embodiments thereof, illustrated by way of non-limiting example in the accompanying drawings, wherein:

FIG. 1 shows a diagram of the operating principle of a device according to the invention;

FIG. 2 shows a block diagram relating to part of the components and to the interaction between this part of the components, of the device as in the diagram of FIG. 1

FIG. 3 shows a further operating diagram of the device of FIG. 1 highlighting a block diagram of its components;

FIGS. 4 and 5 respectively represent two possible embodiments of the optical unit of the device of FIG. 1, shown in schematic form;

FIG. 6 shows a schematic side view of a device according to the invention, in which the flat shape of the device can be appreciated;

FIG. 7 shows an example of device according to the invention also comprising a cart that can move on the ground;

FIG. 8 shows a different embodiment of the invention to the one shown in the preceding figures, in which only one infrared light source and one infrared image acquisition camera are present;

FIG. 9 represents an operating diagram of the device as in FIG. 8;

FIG. 10 represents a diagram of the possible combinations of components of an optical unit of a device according to the invention, also in relation to the light sources thereof;

FIG. 11 represents a diagram of the combination of several optical units in a device according to the invention, also in relation to the light sources thereof;

FIG. 12 represents a diagram of the combination of at least two optical units in a device according to the invention as in FIG. 11, used to implement a three-dimensional view of the area to be investigated.

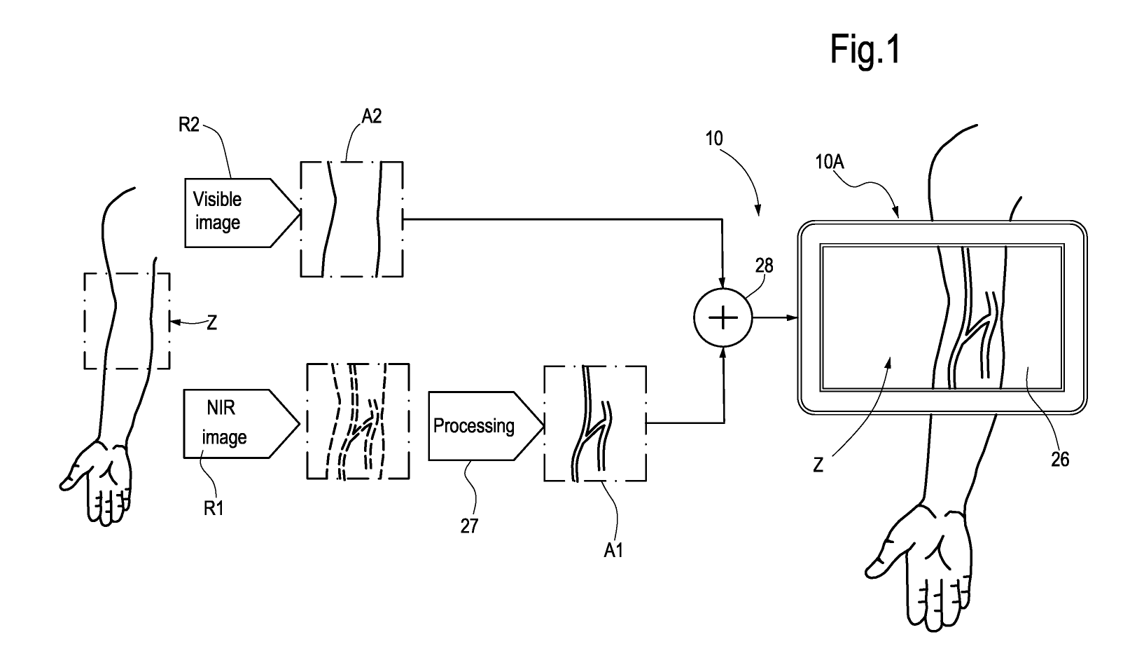

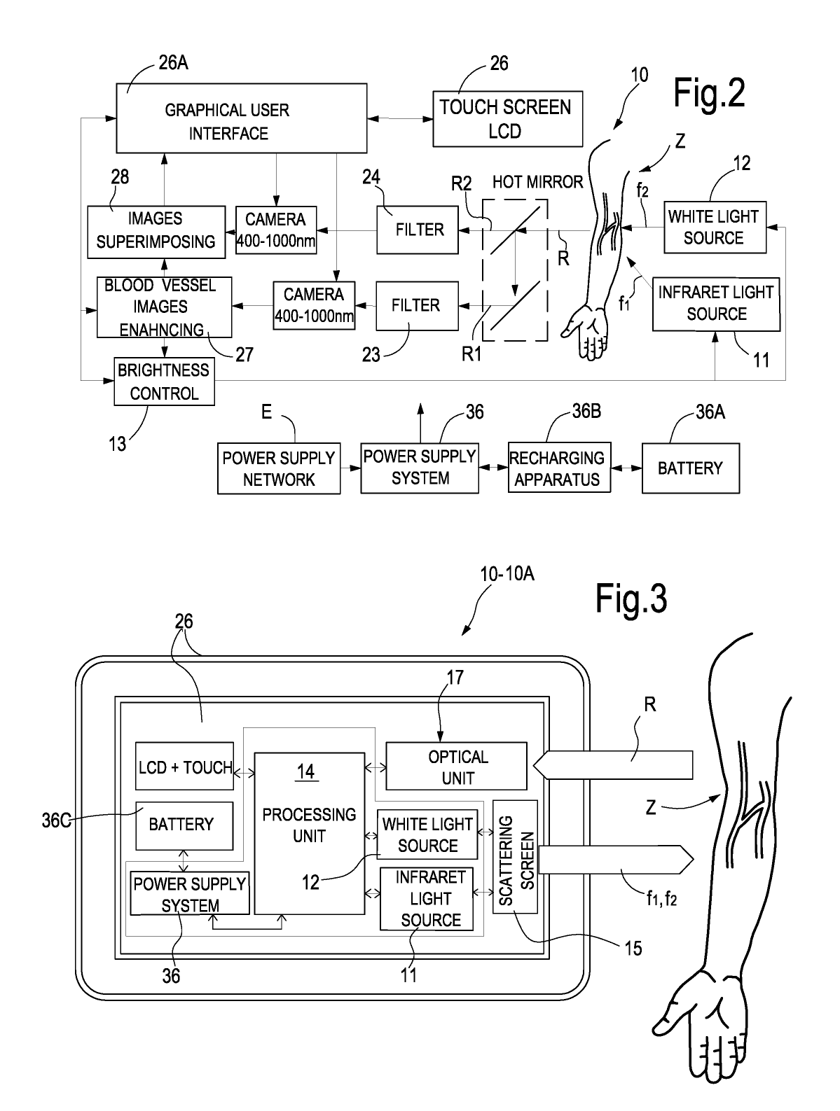

### DETAILED DESCRIPTION OF AN EMBODIMENT OF THE INVENTION

With reference to the aforesaid figures, a device for non-invasive detection of predetermined biological structures of interest according to the invention, hereinafter called hidden biological structures, is indicated as a whole with the number 10. In this example, this device is suitable for detecting, among other things, the surface blood vessels of a patient.

In its main components, the device 10 of this example comprises two light sources, respectively a first source 11 and a second source 12, adapted to illuminate the area Z of the patient being investigated, by means of two light beams with different wavelengths or spectral bands, a first light beam f1 with at least one first wavelength or spectral band selected in a field of wavelengths adapted to be absorbed in a predetermined manner by the hidden biological structure to be detected, in this case the surface venous structure of the area of the patient, and a second light beam f2 in the visible field, adapted to allow viewing in the visible of the area to be investigated in which this venous structure is present.

In the present invention, the term light, light beam, light radiation, optical radiation, optical beam, etc. must be intended as synonyms, i.e. relating to an electromagnetic radiation having components in a given spectral band between 100 nm and 1 cm.

Naturally, in other embodiments, the second source, i.e. the one relating to the light beam with wavelength in the visible, can also be omitted in the case in which ambient light is used to illuminate the area to be investigated.

In this example the first light emitted by the first light source 11, adapted to be absorbed in a predetermined manner by the hidden biological structure to be detected, has a first wavelength equal, for example, to 880 nm, namely selected in a field of wavelengths between approximately 700 nm and 1000000, or preferably between 700 nm and 1000 nm, i.e. approximately in the infrared field. In fact, the hemoglobin present in the venous blood absorbs infrared light.

The second light beam emitted by the second light source 12 is, for example a white light and therefore has a spectrum between approximately 300 nm and 800 nm, i.e. approximately within the visible field.

Preferably, the light sources comprise a planar (or two-dimensional) distribution of LEDs split into at least two groups, at least one first group acting in the infrared field and at least one second group acting in the visible field.

From an operational viewpoint, the first light beam and the second light beam are emitted substantially simultaneously by the respective sources.

In this embodiment, each source is associated with means 13 for adjusting the luminosity/power of the light sources. For example, the LEDs are controlled directly by the processing unit 14 of the device; for example, the luminosity of each group of LEDs is controlled independently. Adjustment of the luminosity is necessary to offset the distance of the device from the patient, the presence of external light and to eliminate any shadows that might be created.

The light generated by the LEDs passes through scattering means 15, such as a scattering screen, which has the purpose of decreasing the directivity of the LEDs and generating more uniform sources.

The device comprises an image acquisition unit 16 with field of view directed at the area to be investigated Z, adapted to acquire images associated respectively with the wavelengths of the light radiation R coming from the area to be investigated Z, and in particular a first image A1 that shows contents relating to the shape and to the arrangement in space of the veins and a second image A2 that has contents relating to the area to be investigated.

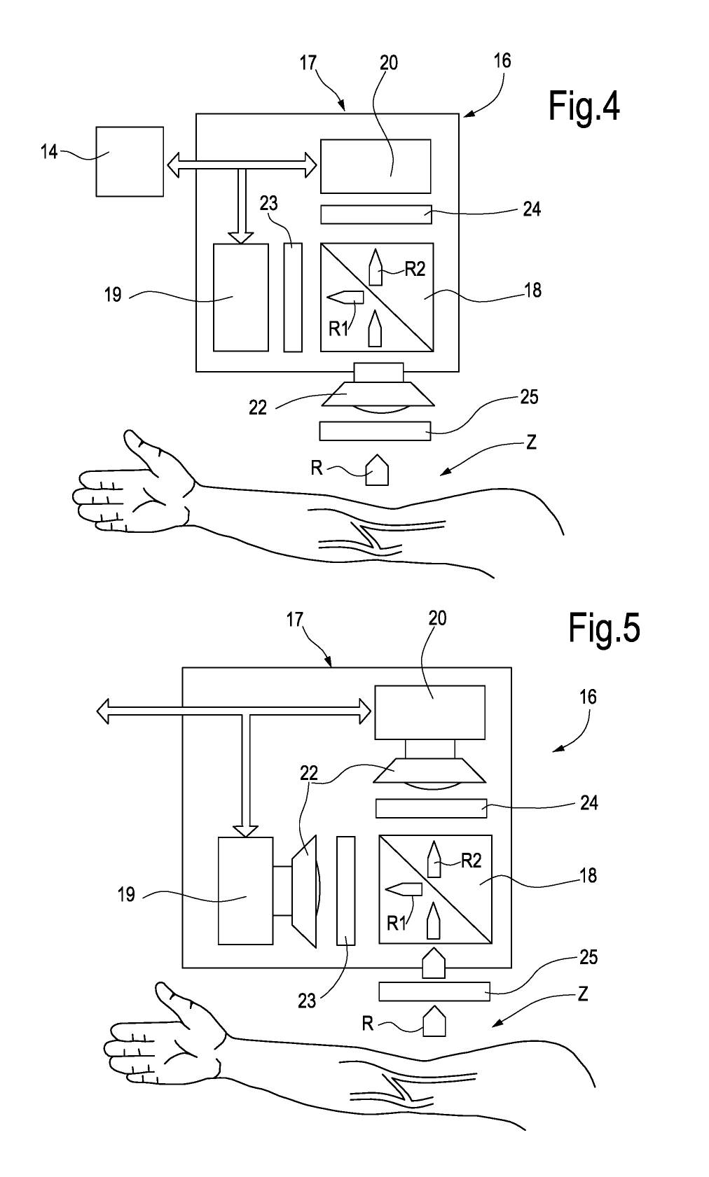

More advantageously (see FIGS. 4 and 5), this image acquisition unit 16 comprises a single optical unit 17 through which the device 10 receives all the light radiation R coming from the area to be investigated Z.

This optical unit 17 comprises splitting means 18 adapted to split the light radiation R coming from the area to be investigated Z, following illumination by the two light sources 11 and 12, into a first and a second light radiation R1 and R2 respectively with wavelength or spectral band corresponding to a field such that a light radiation is capable of being scattered, reflected or absorbed by the hidden biological structure in a manner differing from the surrounding biological structures (or, in the case in question, between 700 nm and 1000000, or preferably between 700 nm and 1000 nm, i.e. in the infrared field) and with wavelength or spectral band included at least in part in the spectral band of the visible or in a band between 300 nm and 800 nm.

Preferably these splitting means 18 comprise a beamsplitter, or the like, of known type, such as a cube beamsplitter or a prism beamsplitter or a three-band beamsplitter, or a hot-mirror, or other means that perform this function.

The same optical unit 17 also comprises two distinct image acquisition cameras, a first camera 19 for acquiring the first image A1 and a second camera 20 for acquiring the second image A2. Advantageously, these cameras are image sensors, such as CCD sensors, CMOS sensors etc.

For example, these image sensors are oriented orthogonally to one another, as the beamsplitter, the hot mirror or the other splitting means 18 allows direct passage of the second light radiation R2, while reflecting the first light radiation in the infrared so as to increase the angle of the optical path for this radiation by 90°. Therefore, the second camera 20 has the receiving surface of the sensor arranged frontally (orthogonally) to the optical axis of the optical unit, while the first camera 19 is arranged oriented through 90° with respect to the former, with the receiving surface of the sensor arranged parallel to the optical axis.

In practice, the beamsplitter, hot mirror or the like is a mirror interposed between optical input of the device and the image receiving unit formed by the two cameras, which allows transmission of the light radiation with wavelength or spectral band in the visible toward the second camera and reflection of the light radiation with wavelength or spectral band in the infrared toward the first camera. The device for splitting the two optical radiations into the two bands considered described above allows the acquisition of the same optical field by the two cameras so that the acquired images A1 (containing information contributions relating to the hidden biological structure) and A2 (containing information contributions relating to viewing the surface of the area to be investigated Z), can be accurately superimposed, if necessary through a simple calibration procedure.

In practice, the optical unit 17 defines optical paths (i.e. routes which from the area Z to be investigated reach corresponding image acquisition cameras 19 and 20), which all have the same optical length, in a known manner.

Preferably, the two image acquisition cameras 19 and 20 comprise respective image sensors sensitive in the same wavelength band between approximately 300 nm and 1000 nm, i.e. encompass both the infrared field and the visible field.

Advantageously, the optical unit 17 also comprises at least one optical focusing system 22. For example, as shown in FIG. 4, this optical focusing system 22 is arranged at the inlet to the optical unit. Alternatively, two optical focusing systems 22 are present, arranged at the inlet to the cameras 19 and 20.

Advantageously, the optical focusing system or systems 22 can comprise a zoom system, to be able to enlarge or reduce the portion of image viewed. This system can advantageously be controlled by the managing system and can be set both automatically (autofocus) and through the indications received by interaction of the user on the monitor of the device or on any other interaction devices.

The optical unit 17 can also comprise a first filter 23 arranged between the beamsplitter, the hot mirror or the like 18, and the first camera 19 that permits the passage, toward said first camera 19, of a light beam with wavelength or spectral band belonging to a field such that a light radiation is capable of being scattered, reflected or absorbed by the hidden biological structure in a manner differing from the surrounding biological structures, preferably approximately in the infrared field between around 700 nm and 1000000 nm, and more preferably between 700 nm and 1000 nm. For example, this first filter has a band of 50 nm centered at the wavelength of 880 nm.

Similarly, the optical unit 17 can also comprise a second filter 24 arranged between the beamsplitter, the hot mirror or the like 18 and the second camera 20 that permits the passage, toward said second camera, of a light beam with wavelength or spectral band belonging approximately to the visible field.

At the inlet to the optical unit 17 there can be present a polarizer 25, especially if the light radiation emitted in the frequencies selected is in turn polarized according to specific directions through suitable polarization devices. This solution can guarantee greater independence of the performance from the external lighting conditions and greater contrast, especially in the image in the infrared field.

It is also possible for the radiations emitted to be discontinuous in time according to a predetermined pattern and for acquisition of the images to be synchronized appropriately with this lighting so as to ensure greater immunity to the variations in luminosity of the surrounding environment.

Naturally, a viewer 26 is present, adapted to allow viewing of the images received by the image receiver.

Preferably, the device can comprise electronic processing means 27 of the first image A1, adapted to increase the contrast or to highlight the shape of the veins, so that, as will be apparent below, this biological structure (the veins) can be clearly distinguished on the viewer 26. The processing algorithms are real time and can follow different strategies and can be based only on the information of the incoming video flow coming from the optical path in the infrared or on all the video flows acquired by the device. This means that in the typical case of two optical paths, one infrared and one in the visible, the processing required to generate the mapping of the biological structures can be based both on the information of the images in the infrared and on that coming from the visible. It goes without saying that there can also be processing means of the image A2 relating to the visible, in order to improve viewing thereof and the information contribution present therein.

According to the invention, the device 10 also comprises electronic means 28 adapted to produce a single image produced by superimposing the first image A1 (information contributions relating to the infrared field) and the second image A2 (information contributions relating to the visible field) obtained from the optical unit and/or subsequently processed, and to transmit this single image to the viewer 26, so that the user of the device, with this device distanced from the area Z to be investigated and looking at the viewer, sees the area to be investigated and, superimposed on this area, the distribution of the veins, in fact producing a viewing device of the type with “augmented reality”.

In this way the operator who, for example, has to take a blood sample from the arm of a patient, through the device with the optical unit aimed at the sample area, clearly and unequivocally sees the external structure of the arm and the distribution of the veins and can safely take the sample, without placing the device in contact with the arm.

In practice, the device consists of an electronic assembly adapted to:
project at least one light beam, with wavelength or spectral band (700÷900 nm) such as to highlight the absorbent behavior of the oxygen-poor hemoglobin (venous blood) with respect to the surrounding tissues; the area to be investigated must be subjected to illumination in the visible, through a light source integrated in the device or through ambient lighting; for example these illuminations are obtained by means of the use of a planar distribution of LEDs;
if necessary use scattered light, polarizing filters or other techniques to reduce the surface reflections that tend to saturate the receiver and reduce the contrast;
receive an image through a single optical unit, separate the backscattered light radiation (i.e. coming from the area to be investigated through reflection and/or scattering) into at least two optical paths having different wavelengths or spectral band (for example 400÷700 and 700÷900) through a hot-mirror, a beamsplitter or other equivalent devices;
propose two identical scenes but with images deriving from radiations with different spectral content through acquisition with two separate image sensors, both sensitive in the band 400-900 nm;
process the images in the infrared field to highlight and then extrapolate only the blood vessels;
view these images superimposed on the images obtained with visible light on an LCD monitor provided with touch-screen in order to allow the operator to interface with the system.

From a structural viewpoint, the device comprises two opposed main faces, a first face 30 on which said viewer 26 is provided and a second opposite face 31 on which the optical inlet 16A of the image acquisition unit 16 is arranged.

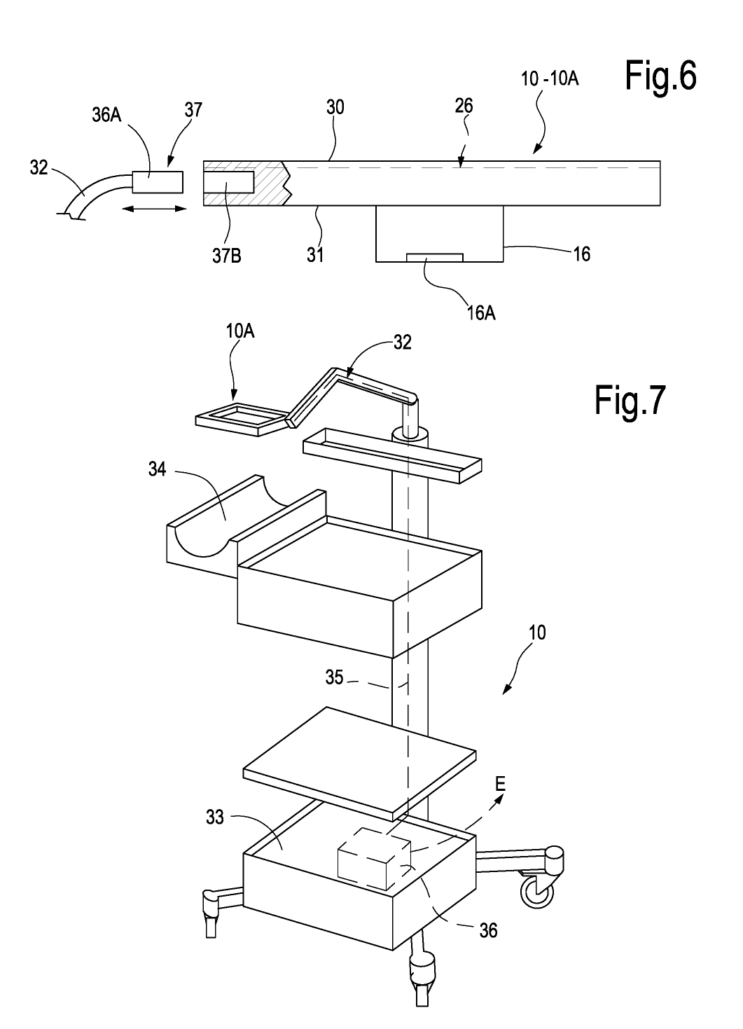

In practice, preferably the majority of the electronic managing, processing and viewing components of the device 10 are substantially comprised between said two faces 30 and 31, as the extension in space of the device is essentially flat, for example similar to the shape of an electronic tablet device. Namely, the viewer comprises a flat display; as can be seen in FIGS. 6, 8 and 11, the optical axis of the optical unit of said image acquisition unit, i.e. the main optical path of the radiation R before being split, is orthogonal to the flat extension of the device; i.e. orthogonal to the plane of the flat display; preferably the optical axis is coincident with an axis passing orthogonally through the center of the display, so as to ensure, in a simple manner, the effect of observation through the device.

Advantageously, the viewer 26 can be a touch-screen monitor and, preferably, the controls for managing the device are prevalently managed by the touch-screen interface of the monitor. The device therefore comprises a graphic interface 26A for the user.

To facilitate use of the device 10, it comprises a supporting arm 32 for connection to a base 33 for supporting the main part 10A of the device comprising the viewer 26 with the image acquisition unit, so that the main part can be positioned stably in space, over the detection area Z.

Preferably, this supporting arm 32 is of jointed type and can comprise one or more motorizations that allow the movement thereof in space, or that allow automated adjustment of the rigidity of the joint. Advantageously, the controls for managing the movement of the arm or stiffening of the joints are implemented on the touch-screen viewer.

The base 33 is preferably a cart that can move on the ground, but alternatively can be a table or any fixed point present in beds, medical equipment or other equipment present in the context in which the device is used. This cart can comprise, among other things, a surface 34 for supporting the body part of the patient with the area to be investigated; for example, this supporting surface 34 has a concavity facing upward and elongated longitudinally to receive a portion of the patient's arm.

Preferably, there is integrated in the arm 32 an electrical connection 35 of the main part to an electrical power source. For example, an electric track or an electric cable connects this main part 10A with the viewer, to a system 36 with battery 36A with recharging apparatus 36B present on the cart and/or with a cable for connection to the power supply network E, or directly to the cable for connection to the power supply network E.

Preferably, reversible connection means 37 are present between the arm 32 and the main part of the device that comprises the viewer 26. Advantageously, these reversible connection means are provided with a connector 37A and a seat 37B for receiving the connector, and an apparatus to lock the connector (not shown in the figures) in said seat that can be released through the action of a specific release button, which can be either mechanical or implemented with electromechanical devices controlled by the processing unit based on interaction of the user on the touch-screen monitor, optionally associated with a password.

Advantageously, to allow the main part 10A of the device to operate independently from the power supply 36 associated with the cart and/or with the power supply network, and therefore to be transported and positioned anywhere, this main part 10A is provided with a rechargeable battery 36C.

The object of the device 10 described here is to view, for example, the surface venous structure of the body and therefore to help medical personnel to identify the ideal position in which to perform, for example, an injection, i.e. to carry out treatments that require precise knowledge of the surface venous structures.

As seen, the device is an instrument mainly created for stationary use but, being equipped with batteries, allows mobile use, at least for a short period of time.

In practice, the device can be used in the same way as a table top magnifying glass, by interposing it between the area to be observed and the operator's eyes, at the distance most comfortable for the user.

The device is equipped with an objective positioned on the lower part that captures the area below and with a viewer positioned on the upper side that reproduces, in real time, the image below.

Using “augmented reality” techniques, the device identifies the position of the veins and superimposes graphic signs in false colors (which represent the veins) on the real image, creating in the user the perception of seeing the veins under the patient's skin.

The device uses as operating principle the characteristic of venous blood to absorb infrared radiation to a greater extent with respect to that of the surrounding tissues to distinguish the vein, not visible to the naked eye, with respect to the surrounding areas.

Therefore, the device is such as to generate an infrared light beam that, striking the area of interest, interacts with the tissue. This light is then reflected and/or scattered with a lower intensity by those areas with a high presence of venous blood, which will therefore be darker.

A sensor sensitive to infrared light is present to receive the image of the area.

To be able also to generate the image representing the surface tissues that act as spatial reference, the device can be provided with a sensor for the visible and, optionally, also with a white light illumination system.

These sensors are inserted in an optic that allows both of them to observe the same field of view in order to generate two superimposable images without parallax errors, regardless of the distance at which the objective is located.

To allow the user to position the device at an optimal distance without any error, the optic is provided with an autofocus system that ensures a detailed image even at distances that are not predetermined.

As said, in addition to this, an automatic or manual system can be provided for adjusting the luminosity of the radiations emitted that enables the optical power to be increased when the viewer is in distal regions to allow overall views of the area of interest and to allow illumination to be decreased during proximal use, preventing glare in the optic and saturation of information in the images acquired.

Another example of device according to the invention with more than two light radiations used and more than two image acquisition sensors, can relate to the detection of both surface venous vessels and arterial vessels. For this purpose, the device has a number of sources such as to provide an optical radiation in the visible band, to reproduce a true image of the tissues surrounding the area to be investigated (as for the example described above) and at least two optical radiations in two different sub-bands in the infrared band; the first sub-band is centered in the absorption spectrum of the oxygenated hemoglobin, the other in the absorption spectrum of the de-oxygenated hemoglobin. Optionally, the device can also comprise a source capable of emitting a fourth radiation in the infrared spectrum linked to the absorption of melanin. The back scattered optical radiation, i.e. coming from the area to be investigated, is captured by at least one optical unit capable of separating the four radiations onto four independent optical paths to reach the same number of cameras capable of acquiring said signals. The four images acquired, which contain corresponding information contributions, are therefore simultaneously acquired and processed through suitable algorithms in order to highlight the position of the veins and of the arteries starting from the respective images, in an adaptive manner taking account of the image with spectral contents relating to the absorption of melanin and of the image in the visible. The results of processing can be merged in real time into a single image with false colors that is capable of highlighting the two vascular systems superimposed on the image deriving from the visible band or can be reproduced separately on the display of the device, and/or on another optional monitor, in different ways, for example placing several images, including those acquired and/or some of those processed, side by side.

Another example of application is linked to the early diagnosis of melanoma in which anatomical aspects of various type can be particularly significant. Currently, size, shape and color as they appear upon simple inspection to the naked eye are the main parameters used to evaluate the nature of the pathology (benign or malignant) or the staging of the pathology, as other parameters particularly important for the prevention or treatment thereof. This analysis can be performed through a device according to the invention capable of illuminating with N optical radiations in bands centered on frequencies and with extensions to be determined that are particularly significant to highlight the aforesaid parameters. For example, a device according to the invention can be provided with a splitter to split the light radiation coming from the investigated area into seven distinct bands, two bands in the ultraviolet, three in the visible and two in the infrared. The device, as indicated above, is capable of splitting the radiations received so that the framed scene is the same, and so as to generate, following acquisition, perfectly superimposable images (if necessary through a calibration step). These images can simply be viewed in real time, enabling the physician to evaluate the shape and the size in spectral bands outside the visible, to obtain information regarding the depth of the neoformation through the images in the infrared bands also scattered from tissue portions below the surface, obtain information regarding the response in absorption in bands at higher frequencies, such as the ultraviolet, and joint information from comparison of these multi-spectral contents. Moreover, the images relating to the infrared bands allow the degree of vascularization of the neoformation to be detected, and if obtained with significant enlargements, the dimension of the vessels that vascularize the investigated tissue can also be detected. This information, obtained directly from the images acquired or following a similar processing to that described above, can be a powerful diagnostic tool for detecting the activity of the neoformation and, consequently, some operating parameters of great help in any surgical or medical procedure. It is also possible to jointly process all the images described so as to extrapolate images, maps or global values of derived parameters that can be more easily related to the stage of development, to the malignancy and to other factors of diagnostic interest. This derived information can be reconstructed through superimposing of the contents in false colors or through any other method of representation that is easily understood by the diagnosing physician.

It is clear that the device according to the invention can therefore be applied to a vast series of diagnostic procedures that relate to other pathologies of the surface tissues, always retaining the possibility of making acquisitions of a same field of view with multiple radiations in different optical bands, of processing them, also jointly, and of viewing them and/or proposing the desired information in an appropriate manner so that the user can obtain the result of the diagnosis in real time on the device or on a suitable viewing means simply by moving the instrument over the area of interest so as to concentrate on the tissues being investigated each time.

It is clear that there can be many examples. In general, it is not necessary to have the same number of sources as the number of cameras that acquire images relating to radiations with different spectral bands and that provide different information contributions relating to radiations with different spectral bands and that provide different information contributions relating to the desired response of the biological structure being analyzed. For example, it is possible to have a number of cameras as a function of the splitting of the information contributions to be isolated from all the light radiation coming from the area to be investigated, i.e. it is possible to split the radiation as a function of sub-bands of a wider band of radiation coming from the area to be investigated, radiation that is a function of the interaction of the same area with the light/lights projected thereon.

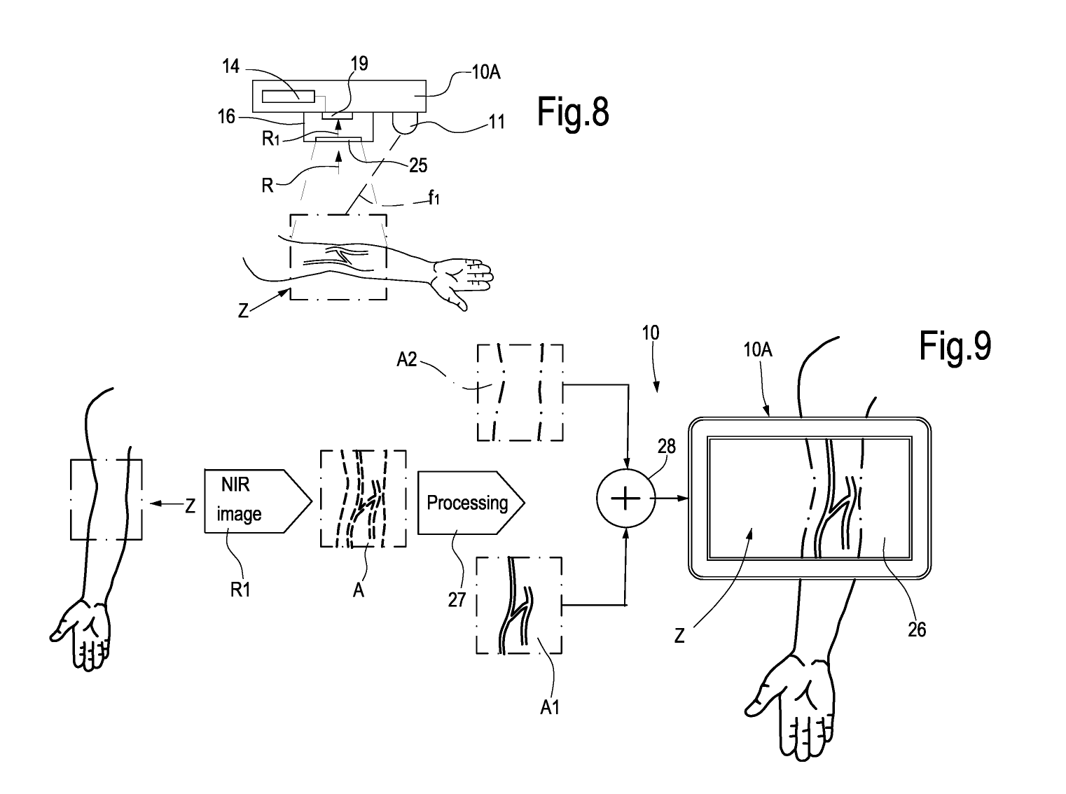

However, it must be noted how in one or the simplest configurations (for example schematized in FIG. 8), the device according to the invention can comprise a single light source 11, for example infrared, and an image acquisition unit 16 formed by a single camera 19 con an image sensor, for example digital, sensitive to the infrared, that will provide an image A; the device emits the infrared light f1, this strikes the area Z to be investigated, for example the body of a patient, and the infrared light interacts with this structure interacting with the cutaneous and subcutaneous tissues. The blood in the veins, which represent a hidden biological structure, absorbs the infrared radiation in a known manner and consequently the light radiation that is reflected and/or scattered and/or emitted from the veins (and that in any case comes from the veins) is a function of known absorption, so that the light radiation that strikes the sensor provides a first group of information contributions relating to this hidden structure that generate a certain part A1 of the image A acquired by the sensor (for example, the shape and the position of the veins and a certain color). The infrared radiation that strikes the patient (in the same field of view as the image acquisition unit) also interacts with biological structures other than the veins, such as the skin surface. The method of this interaction is different with respect to the veins, so that the light radiation that is reflected and/or scattered and/or emitted from the epidermis will be different to that coming from the veins, so that this second light radiation that encounters the sensor provides a second group of information contributions, for example relating to the position and to the shape of the contours of the epidermis, for example defining the surface contours (and/or other surface elements, such as the hairs and other elements associated with the surface visible to the naked eye) of the investigated area (part A2 of the image A). The electronic means of the device then process in 27 the contents relating to the part A1 of the image A, i.e. process the first group of information contributions (the sensor is preferably digital and these information contributions are digital data that form a digital image, and therefore this processing takes place with known image processing techniques), for example to increase the contrast of the shape relating to the veins, and optionally also the contents relating to the part A2 of the image A i.e. the second group of information contributions, are processed in 27 to improve the visibility of the contours of the skin surface (for example the contours of an arm, if this part of the patient's body is being examined). Therefore, the electronic means of the device combine in 28 the two information groups to output an image of the body part being examined with the two biological structures being examined clearly visible, combined with each other but graphically processed optimally for the specific needs, for example the two information groups give rise to two corresponding images that are superimposed on each other to give a single image with the hidden biological structure (for example the veins) clearly defined and with the contours of the body part in which these veins are located clearly recognizable. The image acquisition unit can contain an optical filter 25, for example to exclude the light beams with spectral band outside the desired band, i.e. to transmit to the optical sensor or sensors only the light beams belonging to a given spectral band and therefore obtain images with information contributions associated only with that particular spectral band; for example, the optical filter can be a filter in the band of the infrared and more specifically in the near infrared (NIR).

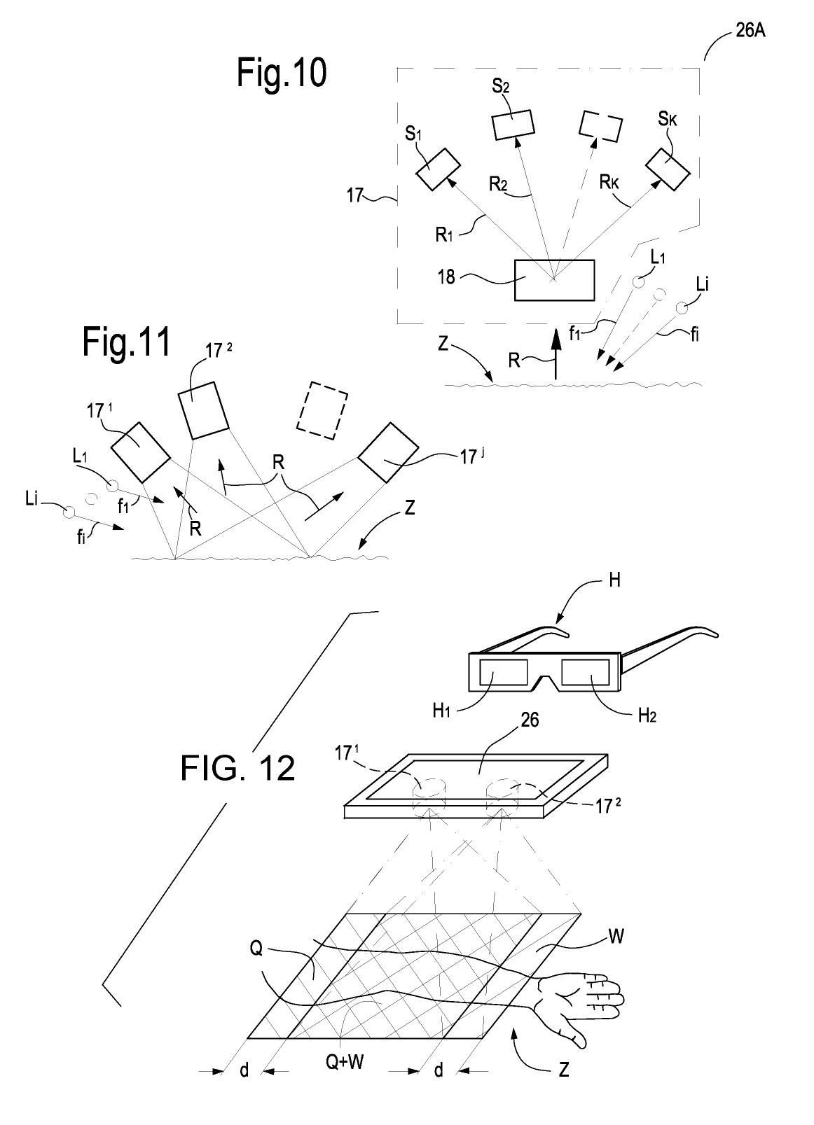

With the exception of the simple case indicated immediately above, in the other examples indicated above reference was made mainly to an image acquisition unit with single optical unit facing the area to be investigated and inside which are splitting means for splitting the radiation into several light beams, each relating to a given spectral band, and relevant image acquisition cameras with information contributions associated with the respective light beams acquired. FIG. 10 shows a general diagram summarizing an optical unit with the possible combinations of device with single optical unit 17, showing a number of cameras S1 . . . Sk variable from two to a number k greater than two, and splitting means 18 for splitting the light beam coming from area to be investigated present in the field of view of the optical unit, in a number of light beams R1 . . . Rk equivalent to the number of the acquisition cameras S1 . . . Sk, each directed at the respective camera. The related acquired images are processed, or not processed, and combined with one another in the most appropriate manner for the type of investigation to be carried out. The device according to the invention can be provided with a number of light sources L1 . . . Li in a quantity variable from one to a number “I” greater than one (to produce light beams f1 . . . fi). The number of sources L and the number of cameras can preferably, but not necessarily, be the same.

In other embodiments, as shown in FIG. 11, there can be present several optical units 17¹ . . . 17^{j}, (variable in a number from two to j, with j greater than two) each with a configuration as described in FIG. 10, with each optical unit provided with the same field of view or approximately the same field of view (for example two nearby fields with a predetermined distance between them). This configuration can allow, if necessary, greater flexibility of use and different types of processing of images and of investigations.

For example, the device according to the invention, which comprises two or more optical units, can be used to perform investigations with augmented reality of 3D type, i.e. to allow stereoscopic viewing of the area to be investigated in relation to each spectral band to be used for analysis of the area.

In practice, the device can be used for non-invasive diagnosis of pathologies of the surface tissues that allows the operator to obtain greater accuracy of the augmented reality experience. This is obtained, for example, by replicating the optical units having the beam splitting means described above in a number of two or more. In this way, it is possible to produce a device that implements the above on at least two superimposed fields of view approximately the same as one another, but centered on two points having a predetermined distance. This allows the acquisition and processing steps to be implemented as described above and, moreover, through appropriate 3D reconstruction algorithms, the scene lying under the instrument to be represented in the correct position in terms of depth, giving the impression of actually observing the tissue being investigated through the instrument and also obtaining the desired information, which is reconstructed through image merging algorithms as described above. This can be obtained through multiple technologies available today such as 3D displays, active or passive glasses and the like. Reference should be made to FIG. 12 for a graphic description of a possible implementation of the system. The figure highlights two optical units 17¹ and 17² with which the fields of view Q and W relating to an area Z are respectively associated. In this example of embodiment, the device acquires, for example, four different images simultaneously (associated with the NIR and visible bands for the optical unit 17¹ relating to the field of view Q and NIR and visible for the optical unit 17² relating to the field of view W) and following processing of the images of the two optical units according to the methods described above, performs a 3D reconstruction of the contributions so as to allow alternate viewing on the display 26. Glasses H (with lenses H1 and H2) allow viewing of the two augmented reality images according to known 3D viewing modes, for example blocking first one lens and then the other: in particular, when the image relating to acquisition and processing deriving from the optical unit 17¹, is proposed on the display 26, the lens H1 will be transparent and the lens H2 will be blacked out. Instead, when the output of the processing of the optical unit 17² is proposed the lens H1 will be blacked out and the lens H2 will be transparent. The glasses H are actively connected to the device in any manner, with or without a transmission cable.

This possible embodiment allows the operator to recover information regarding depth, which can be particularly useful when medical and surgical operations are to be performed directly on the tissue being investigated by viewing it through the monitor of the instrument.

It is understood that the drawing only shows possible non-limiting embodiments of the invention, which can vary in forms and arrangements without however departing from the scope of the concept on which the invention is based. Any reference numerals in the appended claims are provided purely to facilitate the reading thereof, in the light of the above description and accompanying drawings, and do not in any way limit the scope of protection.

1. A device for non-invasive detection of predetermined biological structures, the device comprising:
at least one light source adapted to illuminate an area to be investigated by means of one or more first light beams with spectral bands subject, in a predetermined manner, to absorption and/or reflection and/or to other interaction typical of a particular biological structure to be detected in order to detect at least specific parameters of said biological structure, among which at least a shape and position in space;
an image acquisition unit with field of view directed at said area to be investigated, adapted to acquire images associated with one or more light radiations coming from said area to be investigated, following illumination by said at least one light source and wherein at least a first group of information contributions and at least a second group of information contributions are associated said images, said first group of information contributions relating at least to the biological structure, said first group of information contributions being associated with at least one first light radiation with spectral band corresponding to a field such that a light radiation is capable of being scattered, reflected or absorbed by the biological structure of interest in a manner differing at least in part from surrounding biological tissues and/or structures, so that said images are capable of showing and/or detecting significant parameters of said biological structure of interest, said second group of information contributions differing at least in part from said first group of information contributions, relating to another or other biological structures interacting differently with said first light radiation with respect to said biological structure of interest subjected to investigation, present in a field of view of said image acquisition unit;
an electronic means for processing at least said first group of information contributions relating to the biological structure of interest, and for combining in at least one image processed information contributions of 3 0 said first group of information contributions relating to the biological structure of interest and the information contributions of said second group relating to said another or other biological structures in the field of view of said image acquisition unit;
at least one viewer, adapted to allow viewing of at least said image, so that a user of the device, with said device at a distance from said area to be investigated and observing said at least one viewer, sees continuously at least one processed image of said biological structure of interest combined with an image of said another or other biological structures present in the same field of view.

2. A device according to claim 1, wherein at least a first light source and at least a second light source are present, said at least one said first light source being adapted to illuminate the area to be investigated by means of one or more of said first light beams, at least one said second light source for one or more second light beams with spectral bands differing at least in part from bands of said first light beams emitted by said first light source, said second light beams being adapted to interact optically with said area to be investigated in a different manner with respect to said first light beams and to produce images containing said second group of information contributions, wherein said light beams of said first light source and said second light source are emitted in a substantially simultaneous manner by said first light source and said second light source, wherein the image acquisition unit acquires at least one first image and at least one second image in a substantially simultaneous manner, wherein the device comprises two opposed main faces, a first face on which said at least one viewer is provided and a second opposite face on which an optical inlet of said image acquisition unit is arranged, a majority of electronic managing, processing and viewing components of the device being substantially comprised between said first face and said second opposite face, an extension in space of the device being essentially flat, an optical axis of the optical unit of said image acquisition unit being orthogonal to a flat extension of the device, said at least one viewer comprising a flat display and said optical axis being orthogonal to said flat display, said optical axis being coincident with an axis passing orthogonally through a center of said at least one viewer.

3. A device according to claim 1, wherein the information contributions of said second group of information contributions relate to contours and/or to surface elements of the area to be investigated, such as to define the shape and the position in space of the area to be investigated, so that the images containing said information contributions of said second group are capable of showing a surface contour and/or other surface elements of said area to be investigated.

4. A device according to claim 1, further comprising splitting means for splitting the scattered and/or reflected light beams coming from said area to be investigated, following illumination by said at least one light source, into at least two distinct light radiations, respectively at least one first light radiation with a first spectral band corresponding to a field such that a light radiation is capable of being scattered, reflected or absorbed by the biological structure of interest in a manner differing at least in part from one or more surrounding biological structures, and at least one second light radiation with second spectral band differing at least in part from said first spectral band, capable of being scattered, reflected or absorbed by said biological structure of interest in a manner differing at least in part with respect to said first light radiation, said image acquisition unit being adapted to acquire images respectively associated with said light radiations split by said splitting means, respectively with said at least one first light radiation and with said at least one second light radiation, and at least one first image with information contributions belonging to said first group of information contributions relating to the biological structure of interest and at least one second image with information contributions relating to said second group of information contributions, said electronic means for combining said at least one first image acquired, or a processing of said at least one first image acquired, containing information contributions relating to the biological structure of interest and at least one second image acquired, or a processing of said at least one first image acquired, containing information contributions of said second group of information contributions.

5. A device according to claim 4, wherein said at least one second light radiation has a spectral band included at least in part in the spectral band of a visible field, so that said image acquisition unit is adapted to acquire images respectively associated with said light radiations split by said splitting means, respectively with said at least one first light radiation and with said at least one second light radiation, and at least one first image with information contributions belonging to said first group of information contributions relating to the biological structure of interest and at least one second image with information contributions relating to the surface view of the area to be investigated.

6. A device according to claim 1, wherein said electronic means, adapted to process at least said first group of information contributions relating to hidden biological structure, is adapted to increase/highlight a part relating to the shape of said biological structure of interest, acquisition, processing and combination of said images containing said information contributions of a first image group and second image group taking place in real time.

7. A device according to claim 1, wherein at least a first light source and a second light source are present, respectively, at least one said first light source being adapted to illuminate the area to be investigated by means of one or more of said first light beams, at least one said second light source for one or more second light beams with spectral bands differing at least in part from bands of said first light beams emitted by said first light source, said second light beams being adapted to interact optically with said area to be investigated in a different manner with respect to said first light beams and to produce images containing said second group of information contributions.

8. A device according to claim 7, wherein said second light source emits one or more second light beams with spectral bands at least in a visible field.

9. A device according to claim 7, wherein said light beams of said first light source and said second light source are emitted in a substantially simultaneous manner by said first light source and said second light source.

10. A device according to claim 1, further comprising means for adjusting a luminosity and/or power of said at least one light source.

11. A device according to claim 4, wherein said image acquisition unit comprises at least two distinct image acquisition cameras, at least one first camera to acquire at least one first image deriving from said at least one first light radiation split by said splitting means and associated with viewing of said biological structure of reference, and at least one second camera to acquire at least one second image deriving from said at least one second split light radiation, said at least one second camera being adapted to acquire at least one second image deriving from said at least one second light radiation having spectral band at least in part in a visible field or in a frequency band in which the associated information can be correlated with the viewing of surface biological structures and/or surface elements of the area to be investigated, such as to define the shape and the position in space of the area to be investigated, said at least two distinct image acquisition cameras being image sensors.

12. A device according to claim 11, further comprising at least one optical unit through which to receive the light radiation coming from said area to be investigated, said optical unit comprising said splitting means and said at least two cameras arranged so as to observe the same field of view.

13. A device according to claim 12, wherein said at least one optical unit defines optical paths, which all have the same optical length, said optical paths being routes which from the area to be investigated reach corresponding image acquisition cameras.

14. A device according to claim 12, wherein said at least one optical unit comprises at least one optical focusing system, arranged at an inlet to said optical unit or at an inlet to one or both said cameras.

15. A device according to claim 14, wherein said optical focusing system comprises at least one zoom system.

16. A device according to claim 11, further comprising a first filter arranged between said splitting means and said first camera adapted to permit a passage, toward said at least said first camera, of at least one light beam with spectral band corresponding to a field such that a light radiation is capable of being scattered, reflected or absorbed by hidden biological structure in a manner differing from the surrounding biological structures, approximately in an infrared field between around 700 nm and 1 cm, and more preferably between 700 nm and 1000 nm.

17. A device according to claim 11, further comprising a second filter arranged between said splitting means and said second camera adapted to allow at least one light beam with spectral band belonging to a visible field, to pass toward said second camera.

18. A device according to claim 11, wherein said splitting means adapted to split the light radiation coming from said area comprises at least one mirror adapted to reflect light radiation belonging to a predetermined spectral band toward predetermined image acquisition areas and to transmit the other light radiations belonging to all the other spectral bands toward other image acquisition areas.

19. A device according to claim 11, further comprising at least one single optical unit through which said at least one single optical unit receives all the light radiation coming from said area to be investigated, said optical unit comprising said splitting means and said at least two cameras arranged so as to observe the same field of view.

20. A device according to claim 11, further comprising at least two said optical units facing the same area to be investigated and with fields of view prevalently superimposed, wherein said electronic means is provided with a means for three-dimensional combination of the images obtained from said at least two optical units to perform three-dimensional reconstruction of the area to be investigated, glasses being provided to view on the at least one viewer a three-dimensionality of the area being investigated with the related biological structures of interest.

21. A device according to claim 1, wherein the image acquisition unit acquires said at least one first image and at least one second image in a substantially simultaneous manner.

22. A device according to claim 1, further comprising two opposed main faces, a first face on which said at least one viewer is provided and a second opposite face on which an optical inlet of said image acquisition unit is arranged, a majority of the electronic managing, processing and viewing components of the device are substantially comprised between said two opposed main faces, the extension in space of the device being essentially flat.

23. A device according to claim 22, wherein an optical axis of the optical unit of said image acquisition unit is orthogonal to the extension of the device, said at least one viewer comprising a flat display and said optical axis being orthogonal to said flat display, said optical axis being coincident with an axis passing orthogonally through a center of said at least one viewer.

24. A device according to claim 1, wherein said at least one first light beam emitted by said at least one light source, adapted to be absorbed in a predetermined manner by the biological structure to be detected, has a first spectral band selected in a field of wavelengths between around 700 nm and 1000000 nm, and more preferably between around 700 nm and 1000 nm, approximately in an infrared field, the at least one light beam being emitted by said at least one second light source or emitted by the environment has a spectral band between around 300 nm and 800 nm, approximately in a visible field.

25. A method for non-invasive detection of blood vessels in proximity of the body surface, the method comprising:
illuminating an area to be investigated by means of one or more light beams with spectral bands included in a spectral band subject to absorption and/or to scattering and/or to reflection and/or to another typical optical interaction in a manner predetermined by the biological structure to be detected, and with one or more light beams with a wavelength or spectral band in a visible field;
splitting the light beams scattered and/or reflected and/or interacting with typical types of optical interaction coming from said area to be investigated, following said illumination, into at least two distinct light radiations, respectively at least one first light radiation with spectral band corresponding to a field such that a light radiation is capable of being scattered, reflected or absorbed by the biological structure of interest in a manner differing from the surrounding biological structures, and at least one second light radiation with a spectral band included at least in part in the spectral band of the visible field;
acquiring images respectively associated with said split light radiations, respectively with said at least one first light radiation and with said at least one second light radiation, and at least one first image with information contributions relating to the biological structure of interest and at least one second image with information contributions relating to a surface view of the area to be investigated,
combining said at least one first image acquired, or processing of said at least one first image acquired, containing information contributions relating to the biological structure of interest and at least one second image acquired, or processing of said at least one second image acquired, containing information contributions relating to a surface view of the area to be investigated, to produce a single output image that includes both the image of the information contributions relating to the biological structure of interest, and the image of the information contributions relating to the surface view of the area to be investigated;
viewing said single image or single combined representation properly identified by diagnostic needs on at least one viewer, so that a user observing said at least one viewer sees the area to be investigated and, superimposed on or alternatively proposed in a combined manner to the area, a shape of said biological structure
